(19) United States
(12) Patent Application Publication
(10) Pub. No.: US 2005/0203074 A1
(43) Pub. Date: Sep. 15, 2005

(54) Modified aromatase inhibitors having improved bioavailability

(76) Inventor: Bruce W. Kneller, Randolph, MA (US)

Correspondence Address: LAW OFFICE OF STEVEN B. LEAVITT, 9914 WATERVIEW PARKWAY, ROWLETT, TX, 75089, US

(21) Appl. No.: 11/079,868

(22) Filed: Mar. 12, 2005

Related U.S. Application Data
(60) Provisional application No. 60/552,609, filed on Mar. 12, 2004.

(51) Int. Cl. A61K 31/57

(52) U.S. Cl. 514/178

(57) ABSTRACT

The present invention relates to the modification of 3-beta-hydroxyandrost-4-ene-6,17-dione (“3-OHAT”) and its metabolites/derivatives at either the 3^{rd} or 17^{th} carbon or 3^{rd} and 17^{th} carbons with various ethers and/or esters and to the modification of 3-beta-hydroxyandrost-4-ene-6,17-dione (“3-OHAT”) and its metabolites/derivatives at carbon 6 with a methyl, methoxy, methylene, hydroxymethylene or acetoxy functional group to improve and increase the oral bioavailability and/or plasma half life and/or efficacy in mammals.

This application claims the benefit of U.S. Provisional Application No. 60/552,609, filed Mar. 12, 2004 which is incorporated herein by reference.

### BACKGROUND OF THE INVENTION

1. Technical Field

The present invention relates to hormones that are modified to improve their efficacy. More particularly, the invention relates to the modification of 3-beta-hydroxyandrost-4-ene-6,17-dione (“3-OHAT”) to improve and increase the oral bioavailability and/or plasma half life and/or efficacy in mammals.

2. Description of Related Art

Estrogens are vital to human growth, development, sexual differentiation and other functions. However, excess estrogen may lead to diseases or problems in women including: weight gain, fibrocystic breast disease and breast cancers, certain types of premenstrual syndrome (PMS), migraine headaches, menstrual disturbances and endometriosis. Men may experience infertility and gynecomastia when exposed to excess levels of estrogen.

The enzyme aromatase is utilized for the conversion of androgens into estrogens. Because of its action, pharmaceutical therapies have targed aromatase to decrease estrogen levels in humans. Aromatase inhibitors decrease the levels of estrogen in the body by blocking the conversion of testosterone and certain other androgens into various estrogens. Also, aromatase inhibitors are effective against tumors that depend on estrogen for growth. For instance, the growth of many breast cancers is promoted by estrogen. Most estrogen after menopause, when the ovaries are no longer producing estrogen, is derived from areas outside of the ovaries by the action of aromatase. To counteract the consequences of undesireable estrogren, aromatase inhibitors may be used. The result of which is a treatment for certain forms of breast cancers and estrogen-dependent tumors after menopause. Aromatase inhibitors simply prevent or severely limit aromatizable androgens from being converted to estrogen.

The hormone androst-4-ene-3,6,17-trione (“AT”) is a non-androgenic/non-anabolic steroid that is an irreversible inhibitor (“suicide substrate”) of the enzyme aromatase that is capable of binding directly to aromatase. Currently, AT is sold as an oral dietary supplement for use in humans. By blocking the aromatization of endogenous androgens to estrogens, AT decreases levels of circulating estrogens and increases levels of circulating androgens. AT may be rapidly converted into a 3 beta-reduced metabolite (3-OHAT) with an enzyme other than aromatase in the presence of NADPH (nicotinamide adenine dinucleotide phosphate, reduced form) under either aerobic or anaerobic conditions.

While aromatase inhibitors have been effectively used to decrease circulating levels of estrogen, currently available treatments have substantial room for improvement. Presently available aromatase inhibitors may require frequent and/or large dosing because aromatase inhibitors may be rapidly metabolized by the liver. Additionally, all anti-aromatases cause some increase in potentially undesirable anabolic and androgenic effects however some (e.g. 4-hydroxyandrostenedione or “FORMESTANE®”) have further increased and excessive undesirable anabolic and androgenic effects because their metabolites are inherently anabolic and androgenic, something not seen with 3-OHAT. Such anabolic and androgenic effects include female virilization (hirsutism, clitoralmegaly, deepening of the voice), prostrate enlargement, alopecia, increased cholesterol levels and acne. Existing aromatase inhibitors include anastrazole (Arimidex), exemestane (Aromasin), and letrozole (Femara). Improved anti-aromatase compounds are greatly needed.

### SUMMARY OF THE INVENTION

Bioavailability is defined as the extent to which, and sometimes rate at which, an active moiety (drug or metabolite) enters systemic circulation, thereby gaining access to the site of action. More factors can affect bioavailability when absorption is slow or incomplete than when it is rapid and complete, so slow or incomplete absorption often leads to variable therapeutic responses. Various modifications of 3b-hydroxyandrost-4-ene-6,17-dione (“3-OHAT”) to achieve greater bioavailability and/or plasma half life and/or efficacy are the objects of the present invention.

3-OHAT is an anti-aromatase that offers a multitude of potential modifications without reducing the effectiveness of the desired anti-aromatase properties. All such modifications may alter the bioavailability of the compound to adapt to various delivery methods including oral, buccal, percutaneous, parenteral, or rectal administration. The end result is an improved bioavailability and/or plasma half life and/or efficacy of a modified 3-OHAT to allow for smaller and/or less frequent doses and alternative methods of administration.

### DETAILED DESCRIPTION

When a drug rapidly dissolves and readily permeates membranes, absorption tends to be complete. However, absorption of orally administered drugs is not always complete because of the complexities involved in dissolving and being adsorbed into the blood stream.

Before entering into the peripheral circulation, a drug must move down the GI tract and pass through the integumentary wall and through the liver which are common sites of drug metabolism. Thus, a drug may be metabolized by the liver (first-pass metabolism) into different and potentially inactive molecules before it can be measured in the systemic circulation. Many drugs have low oral bioavailability because of extensive first-pass metabolism. Low bioavailability is most common with oral dosage forms of poorly water-soluble, slowly absorbed drugs.

In humans, androst-4-ene-3,6,17-dione (“AT”) is rapidly metabolized into the 3 beta-reduced metabolite, 3b-hydroxyandrost-4-ene-6,17-dione (“3-OHAT”). Like AT, 3-OHAT has been characterized as a competitive and irreversible inhibitor (suicide inhibitor) of aromatase. It functions by binding to the active site of the aromatase enzyme and preventing the enzyme from interacting with other steroids (including aromatizable androgens like testosterone). 3-OHAT may be used as an anti-aromatic compound in humans and unlike some anti-aromatases (e.g. 4-hydroxyandrostenedione or “FORMESTANE®”) 3-OHAT has no inherent anabolic or androgenic effect, nor do any of its known metabolites.

The use of 3-OHAT over other anti-aromatases presents other benefits. Because of the hydroxyl group in the beta position at carbon 3, the compound may be easily modified. 3-OHAT may be “linked” to various esters (e.g. ethyl carbonate, cypionate, etc.) and ethers (e.g. tetrahydropyranyl, methyl ether, etc.) by its free hydroxyl group at carbon 3 to increase its lipophilicity (fat solubility). Increasing its lipophilicity allows a significant increase in the amount of the active hormone to enter the circulatory system, intact, through the lymphatic system by avoiding the destructive first-pass through the liver. The end result is increased oral bioavailability and half-life which creates greater efficacy.

Such modified forms of 3-OHAT may benefit oral and parenteral administration of the hormone. Because the compound bi-passes the liver and it has an ether or ester at carbon 3, it is metabolized more slowly. Hence, increased bioavailability and/or half life permits treatment with less frequent and/or smaller dosing. As with most other steroidal hormones, micronization also improves bioavailability. Moreover, treatment costs may be less because increased bioavailability and/or half life would permit the hormone to be administered in less frequent and/or smaller doses.

Such modifications may also allow treatment of individuals who are unable to take AT/3-OHAT via the pre-oral route to resort to another route. The more lipophilic modified compound may be used in an oily depot for injection. These modifications to 3-OHAT could also make the molecule more available by buccal, percutaneous, parenteral or rectal administration.

The desired forms of 3-OHAT and many of its metabolites and derivatives may be modified soley at the 3^{rd} or the 17^{th} or modified at both the 3^{rd} and 17^{th} carbon with an ester (e.g. ethyl carbonate, etc.) or an ether (e.g. tetrahydropyranyl, methyl ether, etc.). The 17^{th} carbon can be modified in instances where there is a hydroxyl functional group at carbon #17. 3-OHAT has a ketone at C #17 so there is no place to attach an ether or ester, but some of it's metabolites (e.g. 3b,17b-dihydroxyandrost-4-ene-6-one) do have a hydroxyl at C #17 so they can have an ether or ester at C #3 (like the modification to 3-OHAT), or they can have an ether or ester at C #17 or they can have an ether or ester at C #3 AND C #17 (diester).

There can be monoethers modifications at C #3 and there can be monoethers modifications at C #17 (where allowable, C #17 has a hydroxyl and not a ketone). There can be diethers modifications (where allowable if C #17 has a hydroxyl and not a ketone), but there can not be ether or ester modification to C #19. There should not be ether or ester modification to C #4 even in instances where there is a hydroxyl group at C #4 with the exception that there can be an acetoxy ester at C #4. The modifications do not include molecules with a hydroxymethylene, methylene, methyl, methoxy, acetoxy added at C #6 that also have an ether or ester modification at C #3, C #17 or C #3 and C #17 even if the molecule has a hydroxyl group at C #3 and/or C #17. If C #6 has a hydroxymethylene, methylene, methyl, methoxy, acetoxy added then no ether or ester should be added.

Additionally, modification of 3-OHAT at carbon 6 with a methyl, methoxy, methylene, hydoxymethylene, or acetoxy functional group may also increase oral bioavailability and/or half life and/or efficacy. Desired forms include, but are not limited to:
3b-hydroxy-5a-androstane-6,17-dione;
3b,17b-dihydroxyandrost-4-ene-6-one;
3b,4-dihydroxyandrost-4-ene-6,17-dione;
3b,17b-hydroxy-5a-androstane-6-one;
3b,4-dihydroxy-5a-androstane-6,17-dione;
3b,4,17b-trihydroxyandrost-4-ene-6-one;
3b,4,17b-trihydroxy-5a-androstane-6-one;
5a-androstane-3b,6,17-trione;
3b,19-dihydroxy-5a-androstane-6,17-dione;
3b,17b,19-trihydroxyandrost-4-ene-6-one;
3b,4,19-trihydroxyandrost-4-ene-6,17-dione;
3b,17b,19-trihydroxy-5a-androstane-6-one;
3b,4,19-trihydroxy-5a-androstane-6,17-dione;
3b,4,17b,19-tetrahydroxyandrost-4-ene-6-one;
3b,4,17b,19-tetrahydroxy-5a-androstane-6-one;
19-hydroxy-5a-androstane-3b,6,17-trione;
3b-hydroxy-5a-androstane-6,17,19-trione;
3b,17b-dihydroxyandrost-4-ene-6,19-dione;
3b,4-dihydroxyandrost-4-ene-6,17,19-trione;
3b,17b-hydroxy-5a-androstane-6,19-dione;
3b,4-dihydroxy-5a-androstane-6,17,19-trione;
3b,4,17b-trihydroxyandrost-4-ene-6,19-dione;
3b,4,17b-trihydroxy-5a-androstane-6,19-dione;
5a-androstane-3b,6,17,19-tetrone;
6-methyl-3b-hydroxy-5a-androstane-17-one;
6-methyl-3b,17b-dihydroxyandrost-4-ene;
6-methyl-3b,4-dihydroxyandrost-4-ene-17-one;
6-methyl-3b,17b-hydroxy-5a-androstane;
-6-methyl-3b,4-dihydroxy-5a-androstane-17-one;
6-methyl-3b,4,17b-trihydroxyandrost-4-ene;
6-methyl-3b,4,17b-trihydroxy-5a-androstane;
6-methyl-5a-androstane-3b,17-dione;
6-methoxy-3b-hydroxy-5a-androstane-17-one;
6-methoxy-3b,17b-dihydroxyandrost-4-ene;
6-methoxy-3b,4-dihydroxyandrost-4-ene-17-one;
6-methoxy-3b,17b-hydroxy-5a-androstane;
6-methoxy-3b,4-dihydroxy-5a-androstane-17-one;
6-methoxy-3b,4,17b-trihydroxyandrost-4-ene;
6-methoxy-3b,4,17b-trihydroxy-5a-androstane;
6-methoxy-5a-androstane-3b,17-dione;
6-methylene-3b-hydroxy-5a-androstane-17-one;
6-methylene-3b,17b-dihydroxyandrost-4-ene;
6-methylene-3b,4-dihydroxyandrost-4-ene-17-one;
6-methylene-3b,17b-hydroxy-5a-androstane;
6-methylene-3b,4-dihydroxy-5a-androstane-17-one;
6-methylene-3b,4,17b-trihydroxyandrost-4-ene;
6-methylene-3b,4,17b-trihydroxy-5a-androstane;
6-methylene-5a-androstane-3b,17-dione;
6-hydroxymethylene-3b-hydroxy-5a-androstane-17-one;
6-hydroxymethylene-3b,17b-dihydroxyandrost-4-ene;
-6-hydroxymethylene-3b,4-dihydroxyandrost-4-ene-17-one;
6-hydroxymethylene-3b,17b-hydroxy-5a-androstane;
6-hydroxymethylene-3b,4-dihydroxy-5a-androstane-17-one;
6-hydroxymethylene-3b,4,17b-trihydroxyandrost-4-ene;
6-hydroxymethylene-3b,4,17b-trihydroxy-5a-androstane;
6-hydroxymethylene-5a-androstane-3b,17-dione;
6-acetoxy-3b-hydroxy-5a-androstane-17-one;
6-acetoxy-3b,17b-dihydroxyandrost-4-ene;
6-acetoxy-3b,4-dihydroxyandrost-4-ene-17-one;
6-acetoxy-3b,17b-hydroxy-5a-androstane;
6-acetoxy-3b,4-dihydroxy-5a-androstane-17-one;
6-acetoxy-3b,4,17b-trihydroxyandrost-4-ene;
6-acetoxy-3b,4,17b-trihydroxy-5a-androstane;
6-acetoxy-5a-androstane-3b,17-dione;
4-acetoxy-3b-hydroxyandrost-4-ene-6,17-dione,
4-acetoxy-3b-hydroxy-5a-androstane-6,17-dione;
4-acetoxy-3b,17b-dihydroxyandrost-4-ene-6-one;
4-acetoxy-3b,17b-dihydroxy-5a-androstane-6-one;
4-acetoxy-5a-androstane-3b,6,17-trione;
4-acetoxy-3b,19-dihydroxy-5a-androstane-6,17-dione;
4-acetoxy-3b,17b,19-trihydroxyandrost-4-ene-6-one;
4-acetoxy-3b,19-dihydroxyandrost-4-ene-6,17-dione;
-4-acetoxy-3b,17b,19-trihydroxy-5a-androstane-6-one;
4-acetoxy-3b,19-dihydroxy-5a-androstane-6,17-dione;
4-acetoxy-3b,17b,19-trihydroxyandrost-4-ene-6-one;
4-acetoxy-3b,17b,19-trihydroxy-5a-androstane-6-one;
4-acetoxy-19-hydroxy-5a-androstane-3b,6,17-trione;
4-acetoxy-3b-hydroxy-5a-androstane-6,17,19-trione;
4-acetoxy-3b,17b-dihydroxyandrost-4-ene-6,19-dione;
4-acetoxy-3b-hydroxyandrost-4-ene-6,17,19-trione;
4-acetoxy-3b,17b-hydroxy-5a-androstane-6,19-dione;
4-acetoxy-3b-hydroxy-5a-androstane-6,17,19-trione;
4-acetoxy-3b,17b-dihydroxyandrost-4-ene-6,19-dione;
4-acetoxy-3b,17b-dihydroxy-5a-androstane-6,19-dione.

While the invention has been particularly shown and described with reference to a preferred embodiment, it will be understood by those skilled in the art that various changes in form and detail may be made therein without departing from the spirit and scope of the invention.

1. A hormone for eliciting a desired response comprising at least one modified or derivative of 3b-hydroxyandrost-4-ene-6,17-dione (“3-OHAT”).

2. The hormone according to claim 1, wherein the at least one modified or derivative of 3b-hydroxyandrost-4-ene-6,17-dione (“3-OHAT”) compound is selected from a group comprising:
3b-hydroxy-5a-androstane-6,17-dione;
3b,17b-dihydroxyandrost-4-ene-6-one;
3b,4-dihydroxyandrost-4-ene-6,17-dione;
3b,17b-hydroxy-5a-androstane-6-one;
3b,4-dihydroxy-5a-androstane-6,17-dione;
3b,4,17b-trihydroxyandrost-4-ene-6-one;
3b,4,17b-trihydroxy-5a-androstane-6-one;
5a-androstane-3b,6,17-trione;
3b,19-dihydroxy-5a-androstane-6,17-dione;
3b,17b,19-trihydroxyandrost-4-ene-6-one;
3b,4,19-trihydroxyandrost-4-ene-6,17-dione;
3b,17b,19-trihydroxy-5a-androstane-6-one;
3b,4,19-trihydroxy-5a-androstane-6,17-dione;
3b,4,17b,19-tetrahydroxyandrost-4-ene-6-one;
3b,4,17b,19-tetrahydroxy-5a-androstane-6-one;
19-hydroxy-5a-androstane-3b,6,17-trione;
3b-hydroxy-5a-androstane-6,17,19-trione;
3b,17b-dihydroxyandrost-4-ene-6,19-dione;
3b,4-dihydroxyandrost-4-ene-6,17,19-trione;
3b,17b-hydroxy-5a-androstane-6,19-dione;
3b,4-dihydroxy-5a-androstane-6,17,19-trione;
3b,4,17b-trihydroxyandrost-4-ene-6,19-dione;
3b,4,17b-trihydroxy-5a-androstane-6,19-dione;
5a-androstane-3b,6,17,19-tetrone;
6-methyl-3b-hydroxy-5a-androstane-17-one;
6-methyl-3b,17b-dihydroxyandrost-4-ene;
6-methyl-3b,4-dihydroxyandrost-4-ene-17-one;
6-methyl-3b,17b-hydroxy-5a-androstane;
6-methyl-3b,4-dihydroxy-5a-androstane-17-one;
6-methyl-3b,4,17b-trihydroxyandrost-4-ene;
6-methyl-3b,4,17b-trihydroxy-5a-androstane;
6-methyl-5a-androstane-3b,17-dione;
6-methoxy-3b-hydroxy-5a-androstane-17-one;
6-methoxy-3b,17b-dihydroxyandrost-4-ene;
6-methoxy-3b,4-dihydroxyandrost-4-ene-17-one;
6-methoxy-3b,17b-hydroxy-5a-androstane;
6-methoxy-3b,4-dihydroxy-5a-androstane-17-one;
6-methoxy-3b,4,17b-trihydroxyandrost-4-ene;
6-methoxy-3b,4,17b-trihydroxy-5a-androstane;
6-methoxy-5a-androstane-3b,17-dione;
6-methylene-3b-hydroxy-5a-androstane-17-one;
6-methylene-3b,17b-dihydroxyandrost-4-ene;
6-methylene-3b,4-dihydroxyandrost-4-ene-17-one;
6-methylene-3b,17b-hydroxy-5a-androstane;
6-methylene-3b,4-dihydroxy-5a-androstane-17-one;
6-methylene-3b,4,17b-trihydroxyandrost-4-ene;
6-methylene-3b,4,17b-trihydroxy-5a-androstane;
6-methylene-5a-androstane-3b,17-dione;
6-hydroxymethylene-3b-hydroxy-5a-androstane-17-one;
6-hydroxymethylene-3b,17b-dihydroxyandrost-4-ene;
6-hydroxymethylene-3b,4-dihydroxyandrost-4-ene-17-one;
6-hydroxymethylene-3b,17b-hydroxy-5a-androstane;
6-hydroxymethylene-3b,4-dihydroxy-5a-androstane-17-one;
6-hydroxymethylene-3b,4,17b-trihydroxyandrost-4-ene;
6-hydroxymethylene-3b,4,17b-trihydroxy-5a-androstane;
6-hydroxymethylene-5a-androstane-3b,17-dione;
6-acetoxy-3b-hydroxy-5a-androstane-17-one;
6-acetoxy-3b,17b-dihydroxyandrost-4-ene;
6-acetoxy-3b,4-dihydroxyandrost-4-ene-17-one;
6-acetoxy-3b,17b-hydroxy-5a-androstane;
6-acetoxy-3b,4-dihydroxy-5a-androstane-17-one;
6-acetoxy-3b,4,17b-trihydroxyandrost-4-ene;
6-acetoxy-3b,4,17b-trihydroxy-5a-androstane;
6-acetoxy-5a-androstane-3b,17-dione;
4-acetoxy-3b-hydroxyandrost-4-ene-6,17-dione;
4-acetoxy-3b-hydroxy-5a-androstane-6,17-dione;
4-acetoxy-3b,17b-dihydroxyandrost-4-ene-6-one;
4-acetoxy-3b,17b-dihydroxy-5a-androstane-6-one;
4-acetoxy-5a-androstane-3b,6,17-trione;
4-acetoxy-3b,19-dihydroxy-5a-androstane-6,17-dione;
4-acetoxy-3b,17b,19-trihydroxyandrost-4-ene-6-one;
4-acetoxy-3b,19-dihydroxyandrost-4-ene-6,17-dione;
4-acetoxy-3b,17b,19-trihydroxy-5a-androstane-6-one;
4-acetoxy-3b,19-dihydroxy-5a-androstane-6,17-dione;
4-acetoxy-3b,17b,19-trihydroxyandrost-4-ene-6-one;
4-acetoxy-3b,17b,19-trihydroxy-5a-androstane-6-one;
4-acetoxy-19-hydroxy-5a-androstane-3b,6,17-trione;
4-acetoxy-3b-hydroxy-5a-androstane-6,17,19-trione;
4-acetoxy-3b,17b-dihydroxyandrost-4-ene-6,19-dione;
4-acetoxy-3b-hydroxyandrost-4-ene-6,17,19-trione;
4-acetoxy-3b,17b-hydroxy-5a-androstane-6,19-dione;
4-acetoxy-3b-hydroxy-5a-androstane-6,17,19-trione;
4-acetoxy-3b,17b-dihydroxyandrost-4-ene-6,19-dione;
4-acetoxy-3b,17b-dihydroxy-5a-androstane-6,19-dione.

3. The hormone of claim 2, wherein the modification produces a substantial increase in oral bioavailability and plasma half life in mammals as compared to non-modified 3b-hydroxyandrost-4-ene-6,17-dione (“3-OHAT”) compounds.

4. The hormone of claim 1, wherein the hormone 3b-hydroxyandrost-4-ene-6,17-dione (“3-OHAT”) having a hydroxyl functional group located at carbon 3 is modified at the 3rd carbon by addition of an ether or an ester functional group.

5. The hormone of claim 1, wherein the hormone 3b-hydroxyandrost-4-ene-6,17-dione (“3-OHAT”) is modified at the 6th carbon by addition of a methyl, methoxy, methylene, hydoxymethylene, or acetoxy functional group.

6. The hormone of claim 4, wherein the esters is selected from a group comprising:
formate,
acetate,
propionate,
butyrate,
valerate,
hexanoate,
heptanoate,
octanoate,
nonanoate,
decanoate,
undecanoate,
undecylenate,
cyclohexylmethylcarbonate,
methyl carbonate,
ethyl carbonate,
propyl carbonate,
carbonate,
benzoate,
phenylpropionate,
hemisuccinate,
dichloroacetate,
hexahydrobenzoate,
isobutyrate,
caproate,
isocaproate,
4-methylvalerate,
tosylate,
laurate,
methyl ester,
ethyl ester,
buciclate.

7. The hormone of claim 4, wherein the ethers is selected from a group comprising: tetrahydropyranyl ether, methyl ether, ethyl ether, cyclopenten-1-yloxy ether, cyclopentyl ether, cyclohexyl ether, or methoxycyclopentyl ether.

8. The hormone of claim 1, wherein said hormone 3b-hydroxyandrost-4-ene-6,17-dione (“3-OHAT”) having a hydroxyl functional group located at carbon 17 is modified at the 17th carbon by addition of an ether or an ester functional group.

9. The hormone of claim 7, wherein the esters is selected from a group comprising:
formate,
acetate,
propionate,
butyrate,
valerate,
hexanoate,
heptanoate,
octanoate,
nonanoate,
decanoate,
undecanoate,
undecylenate,
cyclohexylmethylcarbonate,
methyl carbonate,
ethyl carbonate,
propyl carbonate,
carbonate,
benzoate,
phenylpropionate,
hemisuccinate,
dichloroacetate,
hexahydrobenzoate,
isobutyrate,
caproate,
isocaproate,
4-methylvalerate,
tosylate,
laurate,
methyl ester,
ethyl ester,
buciclate.

10. The hormone of claim 7, wherein the ethers is selected from a group comprising:
tetrahydropyranyl ether,
methyl ether,
cyclopenten-1-yloxy ether,
cyclopentyl ether,
cyclohexyl ether,
methoxycyclopentyl ether,
ethyl ether.

11. The hormone of claim 1, wherein said hormone 3b-hydroxyandrost-4-ene-6,17-dione (“3-OHAT”) having a hydroxyl functional group located at carbon 3 and 17 is modified at the 3rd and 17th carbon by addition of an ether or an ester functional group.

12. The hormone of claim 10, wherein the esters is selected from a group comprising:
formate,
acetate,
propionate,
butyrate,
valerate,
hexanoate,
heptanoate,
octanoate,
nonanoate,
decanoate,
undecanoate,
undecylenate,
cyclohexylmethylcarbonate,
methyl carbonate,
ethyl carbonate,
propyl carbonate,
carbonate,
benzoate,
phenylpropionate,
hemisuccinate,
dichloroacetate,
hexahydrobenzoate,
isobutyrate,
caproate,
isocaproate,
4-methylvalerate,
tosylate,
laurate,
methyl ester,
ethyl ester,
buciclate.

13. The hormone of claim 10, wherein the ethers is selected from a group comprising:
tetrahydropyranyl ether,
methyl ether,
cyclopenten-1-yloxy ether,
cyclopentyl ether,
cyclohexyl ether,
methoxycyclopentyl ether,
ethyl ether.

14. A method for using a hormone to elicit a desired response comprising: administering an effective amount of a food supplement or drug having 3b-hydroxyandrost-4-ene-6,17-dione (“3-OHAT”) or metabolites or one or more modified forms of the molecule wherein the modification is at the 3rd carbon, at the 17^{th} carbon, or at both the 3^{rd} and 17^{th} carbon, by addition of an ether or an ester functional group or at the 6^{th} carbon by the addition of a methyl, methoxy, methylene, hydoxymethylene, or acetoxy functional group.

15. The method of claim 14 by buccal, preoral, percutaneous, parenteral or rectal administration.

16. The method of claim 14 wherein the one or more modified 3b-hydroxyandrost-4-ene-6,17-dione (“3-OHAT”) compounds or metabolite compounds produces a substantial increase in oral bioavailability and/or plasma half life and/or efficacy in mammals as compared to non-modified 3b-hydroxyandrost-4-ene-6,17-dione (“3-OHAT”).

17. The method of claim 14 wherein the desired response includes inhibition of the aromatase enzyme or a decrease in circulating levels of estrogens or an increase in circulating levels of androgens or combinations thereof.

18. The method of claim 14 wherein 3b-hydroxyandrost-4-ene-6,17-dione (“3-OHAT”) or metabolites or a modified version of the molecule is administered via cyclodextran, liposomal or polyethyleneglycol (PEG) solution.

19. A method for using a hormone to elicit a desired response comprising: administering an effective amount of a food supplement or drug consisting of a metabolite or modified form of 3b-hydroxyandrost-4-ene-6,17-dione (“3-OHAT”) having a hydroxyl functional group at carbon 17 wherein the modification is at the 17th carbon, by addition of an ether or an ester group.

20. The method of claim 19 by buccal, preoral, percutaneous, parenteral or rectal administration.

21. The method of claim 19 wherein the one or more modified 3b-hydroxyandrost-4-ene-6,17-dione (“3-OHAT”) metabolite or modified compounds produces a substantial increase in oral bioavailability and/or plasma half life and/or efficacy in mammals as compared to non-modified 3b-hydroxyandrost-4-ene-6,17-dione (“3-OHAT”).

22. The method of claim 19 wherein the desired response includes inhibition of the aromatase enzyme or a decrease in circulating levels of estrogens or an increase in circulating levels of androgens or combinations thereof.

23. The method of claim 19 wherein the compound produces a substantial increase in oral bioavailability and/or plasma half life and/or efficacy in mammals as compared to the non-modified 3b-hydroxyandrost-4-ene-6,17-dione (“3-OHAT”) compound or metabolites.

24. The method of claim 19 wherein 3b-hydroxyandrost-4-ene-6,17-dione (“3-OHAT”) or metabolites or a modified version of the molecule is administered via cyclodextran, liposomal or polyethyleneglycol (PEG) solution.

25. A method for using a hormone to elicit a desired response comprising: administering an effective amount of a food supplement or drug consisting of a metabolite or modified form of 3b-hydroxyandrost-4-ene-6,17-dione (“3-OHAT”) having a hydroxyl functional group at carbon 17 wherein the modification is at the 3rd and 17th carbon, by addition of an ether or an ester functional group.

26. The method of claim 25 by buccal, preoral, precutaneous, parenteral or rectal administration.

27. The method of claim 25 wherein the one or more modified 3b-hydroxyandrost-4-ene-6,17-dione (“3-OHAT”) compounds or metabolite compounds produces a substantial increase in oral bioavailability and/or plasma half life and/or efficacy in mammals as compared to non-modified 3b-hydroxyandrost-4-ene-6,17-dione (“3-OHAT”).

28. The method of claim 25 wherein the desired response includes inhibition of the aromatase enzyme or a decrease in circulating levels of estrogens or an increase in circulating levels of androgens or combinations thereof.

29. The method of claim 25 wherein 3b-hydroxyandrost-4-ene-6,17-dione (“3-OHAT”) or metabolites or a modified version of the molecule is administered via cyclodextran, liposomal or polyethyleneglycol (PEG) solution.

30. A method for using a hormone to elicit a desired response by administering an effective amount of a supplement having at least one modified 3b-hydroxyandrost-4-ene-6,17-dione (“3-OHAT”) compound selected from the group comprising:
3b-hydroxy-5a-androstane-6,17-dione;
3b,17b-dihydroxyandrost-4-ene-6-one;
3b,4-dihydroxyandrost-4-ene-6,17-dione;
3b,17b-hydroxy-5a-androstane-6-one;
3b,4-dihydroxy-5a-androstane-6,17-dione;
3b,4,17b-trihydroxyandrost-4-ene-6-one;
3b,4,17b-trihydroxy-5a-androstane-6-one;
5a-androstane-3b,6,17-trione;
3b,19-dihydroxy-5a-androstane-6,17-dione;
3b,17b,19-trihydroxyandrost-4-ene-6-one;
3b,4,19-trihydroxyandrost-4-ene-6,17-dione;
3b,17b,19-trihydroxy-5a-androstane-6-one;
3b,4,19-trihydroxy-5a-androstane-6,17-dione;
3b,4,17b,19-tetrahydroxyandrost-4-ene-6-one;
3b,4,17b,19-tetrahydroxy-5a-androstane-6-one;
19-hydroxy-5a-androstane-3b,6,17-trione;
3b-hydroxy-5a-androstane-6,17,19-trione;
3b,17b-dihydroxyandrost-4-ene-6,19-dione;
3b,4-dihydroxyandrost-4-ene-6,17,19-trione;
3b,17b-hydroxy-5a-androstane-6,19-dione;
3b,4-dihydroxy-5a-androstane-6,17,19-trione;
3b,4,17b-trihydroxyandrost-4-ene-6,19-dione;
3b,4,17b-trihydroxy-5a-androstane-6,19-dione;
5a-androstane-3b,6,17,19-tetrone;
6-methyl-3b-hydroxy-5a-androstane-17-one;
6-methyl-3b,17b-dihydroxyandrost-4-ene;
6-methyl-3b,4-dihydroxyandrost-4-ene-17-one;
6-methyl-3b,17b-hydroxy-5a-androstane;
6-methyl-3b,4-dihydroxy-5a-androstane-17-one;
6-methyl-3b,4,17b-trihydroxyandrost-4-ene;
6-methyl-3b,4,17b-trihydroxy-5a-androstane;
6-methyl-5a-androstane-3b,17-dione;
6-methoxy-3b-hydroxy-5a-androstane-17-one;
6-methoxy-3b,17b-dihydroxyandrost-4-ene;
6-methoxy-3b,4-dihydroxyandrost-4-ene-17-one;
6-methoxy-3b,17b-hydroxy-5a-androstane;
6-methoxy-3b,4-dihydroxy-5a-androstane-17-one;
6-methoxy-3b,4;17b-trihydroxyandrost-4-ene;
6-methoxy-3b,4,17b-trihydroxy-5a-androstane;
6-methoxy-5a-androstane-3b,17-dione;
6-methylene-3b-hydroxy-5a-androstane-17-one;
6-methylene-3b,17b-dihydroxyandrost-4-ene;
6-methylene-3b,4-dihydroxyandrost-4-ene-17-one;
6-methylene-3b,17b-hydroxy-5a-androstane;
6-methylene-3b,4-dihydroxy-5a-androstane-17-one;
6-methylene-3b,4,17b-trihydroxyandrost-4-ene;
6-methylene-3b,4,17b-trihydroxy-5a-androstane;
6-methylene-5a-androstane-3b,17-dione;
6-hydroxymethylene-3b-hydroxy-5a-androstane-17-one;
6-hydroxymethylene-3b,17b-dihydroxyandrost-4-ene;
6-hydroxymethylene-3b,4-dihydroxyandrost-4-ene-17-one;
6-hydroxymethylene-3b,17b-hydroxy-5a-androstane;
6-hydroxymethylene-3b,4-dihydroxy-5a-androstane-17-one;
6-hydroxymethylene-3b,4,17b-trihydroxyandrost-4-ene;
6-hydroxymethylene-3b,4,17b-trihydroxy-5a-androstane;
6-hydroxymethylene-5a-androstane-3b,17-dione;
6-acetoxy-3b-hydroxy-5a-androstane-17-one;
6-acetoxy-3b,17b-dihydroxyandrost-4-ene;
6-acetoxy-3b,4-dihydroxyandrost-4-ene-17-one;
6-acetoxy-3b,17b-hydroxy-5a-androstane;
6-acetoxy-3b,4-dihydroxy-5a-androstane-17-one;
6-acetoxy-3b,4,17b-trihydroxyandrost-4-ene;
6-acetoxy-3b,4,17b-trihydroxy-5a-androstane;
6-acetoxy-5a-androstane-3b,17-dione;
4-acetoxy-3b-hydroxyandrost-4-ene-6,17-dione;
4-acetoxy-3b-hydroxy-5a-androstane-6,17-dione;
4-acetoxy-3b,17b-dihydroxyandrost-4-ene-6-one;
4-acetoxy-3b,17b-dihydroxy-5a-androstane-6-one;
4-acetoxy-5a-androstane-3b,6,17-trione;
4-acetoxy-3b,19-dihydroxy-5a-androstane-6,17-dione;
4-acetoxy-3b,17b,19-trihydroxyandrost-4-ene-6-one;
4-acetoxy-3b,19-dihydroxyandrost-4-ene-6,17-dione;
4-acetoxy-3b,17b,19-trihydroxy-5a-androstane-6-one;
4-acetoxy-3b,19-dihydroxy-5a-androstane-6,17-dione;
4-acetoxy-3b,17b,19-trihydroxyandrost-4-ene-6-one;
4-acetoxy-3b,17b,19-trihydroxy-5a-androstane-6-one;
4-acetoxy-19-hydroxy-5a-androstane-3b,6,17-trione;
4-acetoxy-3b-hydroxy-5a-androstane-6,17,19-trione;
4-acetoxy-3b,17b-dihydroxyandrost-4-ene-6,19-dione;
4-acetoxy-3b-hydroxyandrost-4-ene-6,17,19-trione;
4-acetoxy-3b,17b-hydroxy-5a-androstane-6,19-dione;
4-acetoxy-3b-hydroxy-5a-androstane-6,17,19-trione;
4-acetoxy-3b,17b-dihydroxyandrost-4-ene-6,19-dione;
4-acetoxy-3b,17b-dihydroxy-5a-androstane-6,19-dione.

31. The method of claim 30 by buccal, preoral, precutaneous, parenteral or rectal administration.

32. The method of claim 30 wherein the one or more modified 3b-hydroxyandrost-4-ene-6,17-dione (“3-OHAT”) compounds or metabolite compounds produces a substantial increase in oral bioavailability and/or plasma half life and/or efficacy in mammals as compared to non-modified 3b-hydroxyandrost-4-ene-6,17-dione (“3-OHAT”).

33. The method of claim 30 wherein the desired response includes inhibition of the aromatase enzyme or a decrease in circulating levels of estrogens or an increase in circulating levels of androgens or combinations thereof.

34. The method of claim 30 wherein the compound produces a substantial increase in oral bioavailability and/or plasma half life and/or efficacy in mammals as compared to the non-modified 3b-hydroxyandrost-4-ene-6,17-dione (“3-OHAT”) compound or metabolites.

35. The method of claim 30 wherein 3b-hydroxyandrost-4-ene-6,17-dione (“3-OHAT”) or metabolites or a modified version of the molecule is administered via cyclodextran, liposomal or polyethyleneglycol (PEG) solution.

36. A hormone for eliciting a desired response comprising at least one modified or derivative of 3b-hydroxyandrost-4-ene-6,17-dione (“3-OHAT”) that is modified at carbon 6 with the addition of a methyl, methoxy, methylene, hydroxymethylene or acetoxy functional groups.

37. The hormone of claim 36, wherein the modification produces a substantial increase in oral bioavailability and plasma half life in mammals as compared to non-modified 3b-hydroxyandrost-4-ene-6,17-dione (“3-OHAT”) compounds.

38. A hormone for eliciting a desired response comprising at least one modified or derivative of 3b-hydroxyandrost-4-ene-6,17-dione (“3-OHAT”) that is modified at carbon 4 with an acetoxy functional group.

39. The hormone of claim 38, wherein the modification produces a substantial increase in oral bioavailability and plasma half life in mammals as compared to non-modified 3b-hydroxyandrost-4-ene-6,17-dione (“3-OHAT”) compounds.
